(19) United States
(12) Patent Application Publication
(10) Pub. No.: US 2021/0257071 A1
(43) Pub. Date: Aug. 19, 2021

(54) Health Monitoring Method and System, Print Terminal, and Computer-Readable Storage Medium

(71) Applicant: BOE TECHNOLOGY GROUP CO., LTD., Beijing (CN)

(72) Inventors: Xu Zhang, Beijing (CN); Xin Wei, Beijing (CN); Ping Jin, Beijing (CN); Qingbing Liu, Beijing (CN)

(21) Appl. No.: 16/649,552

(22) PCT Filed: Apr. 8, 2019
(86) PCT No.: PCT/CN2019/081723

§ 371 Date: Mar. 20, 2020

(51) Int. Cl. G16H 15/00; G06K 1/12; G06K 19/06; G16H 10/60; G16H 50/30; G16H 40/67; G06F 3/12

(52) U.S. Cl. CPC G16H 15/00; G06K 1/121; G06K 19/06037; G06F 3/1243; G16H 10/60; G16H 50/30; G16H 40/67; G06K 19/06112

(57) ABSTRACT

The present disclosure relates to a health monitoring method and system, a print terminal, and a computer-readable storage medium. The print terminal includes: a communication device configured to acquire detected physical parameter information; a processor configured to generate a data two-dimensional code comprising the physical parameter information; and a print device configured to print the data two-dimensional code.

### TECHNICAL FIELD

The present disclosure relates to the field of computer technology, and in particular, to a health monitoring method and system, a print terminal, and a computer-readable storage medium.

### BACKGROUND

Two-dimensional code technology is widely used in various fields due to its advantages such as rapid identification, reliability, and low cost.

The related art discloses an electronic scale provided with a two-dimensional code generating device, capable of converting measurement data of an user into a two-dimensional code. The user can obtain a measurement report by scanning the two-dimensional code with a mobile phone.

### SUMMARY

According to a first aspect of the embodiments of the present disclosure, there is provided a print terminal comprising: a communication device configured to acquire detected physical parameter information; a processor configured to generate a data two-dimensional code comprising the physical parameter information; and a printing device configured to print the data two-dimensional code.

In some embodiments, the print terminal further comprises an input device configured to obtain basic user information, wherein the generated data two-dimension code comprises the basic user information.

In some embodiments, the print terminal further comprises: a display device configured to display the data two-dimensional code.

In some embodiments, the print device is configured to print a detection report comprising the physical parameter information.

In some embodiments, the print terminal further comprises: a memory configured to store the acquired basic user information and the physical parameter information.

In some embodiments, the print terminal is a handheld smart printer.

According to a second aspect of the embodiments of the present disclosure, there is provided a health monitoring system, comprising the print terminal according to any one of the above embodiments.

In some embodiments, the health monitoring system further comprises at least one physical parameter detection device configured to detect a human body to obtain the physical parameter information.

In some embodiments, the at least one physical parameter detection device comprises a plurality of physical parameter detection devices configured to obtain different physical parameter information respectively; and the processor of the print terminal is configured to generate different data two-dimensional codes for different physical parameter information.

In some embodiments, the at least one physical parameter detection device comprises a plurality of physical parameter detection devices configured to obtain different physical parameter information respectively; and the processor of the print terminal is configured to generate a data two-dimensional code comprising different physical parameter information.

In some embodiments, the display device of the print terminal is configured to display a device two-dimensional code of the at least one physical parameter detection device.

In some embodiments, the communication device of the print terminal communicates with the at least one physical parameter detection device via Bluetooth.

In some embodiments, the processor of the print terminal is configured to control an operating mode of the at least one physical parameter detection device in response to a user's selection.

In some embodiments, the health monitoring system further comprises: a server configured to process the physical parameter information provided by the user terminal based on the data two-dimensional code, and generate a health report.

In some embodiments, the server is further configured to: create a user account according to the basic user information provided by the user terminal based on the data two-dimensional code, and record physical parameter information corresponding to the user account.

According to a third aspect of the embodiments of the present disclosure, there is provided a health monitoring method, comprising: acquiring detected physical parameter information; generating a data two-dimensional code comprising the physical parameter information; and printing the data two-dimensional code.

In some embodiments, the health monitoring method further comprises displaying the data two-dimensional code in response to a user's request.

In some embodiments, the health monitoring method further comprises in response to a user's selection, controlling an operating mode of the physical parameter detection device for obtaining the physical parameter information.

In some embodiments, the health monitoring method further comprises processing the physical parameter information provided by the user terminal based on the data two-dimensional code, and generating a health report.

According to a fourth aspect of the embodiments of the present disclosure, there is provided a computer-readable storage medium on which a computer program is stored, the program, when executed by a processor, implementing the display method according to any of the foregoing embodiments.

Other features of the present disclosure and advantages thereof will become apparent from the following detailed description of the embodiments of the present disclosure with reference to the accompanying drawings.

### BRIEF DESCRIPTION OF THE DRAWINGS

The accompanying drawings which constitute a part of the specification describe the embodiments of this disclosure, and together with the description, serve to explain the principle of this disclosure.

This disclosure can be understood more clearly with reference to the accompanying drawings according to the following detailed description, in which:

FIG. 1A is a block diagram illustrating a print terminal according to an embodiment of the present disclosure;

FIG. 1B is a block diagram illustrating a print terminal according to another embodiment of the present disclosure;

FIG. 2 is a block diagram illustrating a health monitoring system according to an embodiment of the present disclosure;

FIG. 3 is a block diagram illustrating a health monitoring system according to another embodiment of the present disclosure;

FIG. 4A is a flowchart illustrating a health monitoring method according to an embodiment of the present disclosure;

FIG. 4B is a flowchart illustrating a health monitoring method according to another embodiment of the present disclosure;

FIG. 5 is a block diagram illustrating an electronic device according to an embodiment of the present disclosure;

FIG. 6 is a block diagram illustrating a computer system for implementing some embodiments of the present disclosure.

It should be noted that, the dimensions of the parts shown in the accompanying drawings are not drawn in accordance with actual proportional relationships. In addition, identical or similar reference numerals represent identical or similar composite parts.

### DETAILED DESCRIPTION

The various exemplary embodiments of this disclosure are now described in detail with reference to the accompanying drawings. The description of the exemplary embodiment is merely illustrative and by no means serves as any restriction to this disclosure and its application or use. This disclosure can be implemented in many different forms and is not limited to the embodiments described here. These embodiments are provided in order to make this disclosure thorough and complete, and to fully express the scope of this disclosure to a person skilled in the art. It should be noted that, unless otherwise specified, the relative arrangements of the components and steps described in these embodiments should be interpreted as merely illustrative but not restrictive.

The use of “first,” “second,” and similar words in this disclosure is not intended to indicate any order, quantity, or importance, but rather is used to distinguish one element from another. The word “comprising” or the like means that the elements preceding the word encompass the elements listed after the word and do not exclude the possibility that other elements are also encompassed. “upper”, “lower”, “left”, “right”, and the like are used only to indicate relative position relations, and when an absolute position of the object being described is changed, the relative position relations may also be changed accordingly.

In the present disclosure, when a specific element is described as being positioned between a first element and a second element, there may or may not be an intervening element between the specific element and the first element or the second element.

All terms (comprising technical terms or scientific terms) that are used in this disclosure have the same meanings as those understood by a person of ordinary skill in the field to which this disclosure pertains, unless otherwise specifically defined. It should also be understood that, terms defined in common dictionaries should be interpreted as having meanings consistent with their meanings in the context of the related art, rather than being interpreted in an idealized or extremely formalized sense, unless expressly defined here.

The technologies, methods and apparatuses known to those skilled in the related fields may not be discussed in detail, but where appropriate, the techniques, methods and apparatuses should be considered as part of the specification.

In related technologies, the acquisition of a measurement report based on a two-dimensional code is limited by the need of providing a two-dimensional code generating device for the measurement device, which has a high cost, and is not flexible and convenient.

In view of this, the present disclosure provides a health monitoring method capable of acquiring health detection information more flexibly and conveniently.

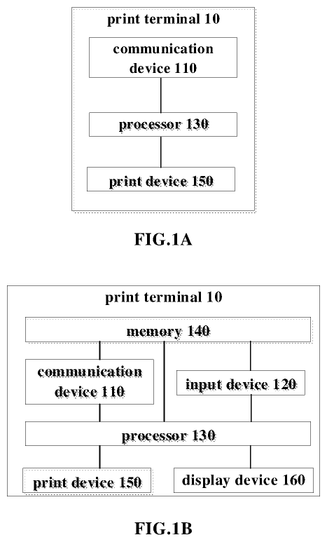

FIG. 1A is a block diagram illustrating a print terminal according to some embodiments of the present disclosure.

As shown in FIG. 1A, the print terminal 10 comprises a communication device 110, a processor 130, and a print device 150.

The communication device 110 is configured to acquire detected physical parameter information.

The physical parameter information can be obtained by detecting a human body with a physical parameter detection device. The physical parameter information comprises one or more of a human blood-gas parameter, a hemodynamic parameter, a blood chemical parameter, a weight parameter, a height parameter, a body fat parameter, a moisture parameter, a bone parameter, a blood pressure parameter, a blood lipid parameter, a heart rate parameter, and a plasma glucose parameter.

The communication device 110 communicates with the physical parameter detection device through a network, such as a wireless network, a wired network, and/or any combination of a wireless network and a wired network. The network may comprise a local area network, the Internet, a telecommunications network, the Internet of Things based on the Internet and/or a telecommunications network, and/or any combination of the above networks, so as to obtain physical parameter information from the physical parameter detection device. A wired network can use, for example, twisted pair, coaxial cable, or fiber optic transmission for communication, and a wireless network can use, for example, a 3G/4G/5G mobile communication network, Bluetooth, Zigbee, or Wi-Fi for communication. In some embodiments, the communication device communicates with the physical parameter detection device via Bluetooth.

The processor 130 is configured to generate a data two-dimensional code comprising the physical parameter information. In some embodiments, the processor 130 comprises a two-dimensional code generator for converting the physical parameter information into a data two-dimensional code.

The print device 150 is configured to print the data two-dimensional code. In some embodiments, the print device 150 is further configured to print a detection report comprising physical parameter information. The print device 150 may print a data two dimensional code or a detection report according to a user's selection.

FIG. 1B is a block diagram illustrating a print terminal according to other embodiments of the present disclosure. FIG. 1B is different from FIG. 1A in that the print terminal 10′ in FIG. 1B further comprises at least one of an input device 120, a memory 140, or a display device 160. Only the difference between FIG. 1B and FIG. 1A will be described below, and the same as in FIG. 1A will not be repeated.

As shown in FIG. 1B, in some embodiments, the print terminal 10′ further comprises an input device 120. The input device 120 is configured to obtain basic user information.

The input device 120 comprises a keyboard, a microphone, a scanner, a camera, etc. The basic user information comprises, for example, name, gender, age, ID number, and so on. In some embodiments, the basic user information is obtained by manual entry of the user. In other embodiments, the basic user information is obtained by scanning the user's ID card or health insurance card.

In addition to the physical parameter information, the two-dimensional code generated by the processor 130 may comprise basic user information obtained through the input device 120.

In some embodiments, the print terminal 10′ further comprises a memory 140. The memory 140 is configured to store the acquired basic user information and physical parameter information. The stored basic user information and physical parameter information can be used as historical records for retrieval.

In other embodiments, the print terminal 10′ further comprises a display device 160. The display device 160 may display a data two-dimensional code comprising the physical parameter information. The display device 160 may also display a detection report comprising the physical parameter information.

It should be understood that the input device 120, the memory 140, and the output device 160 may not be components of the print terminal, but may be connected to the print terminal through a network. The print terminal is, for example, a handheld smart printer.

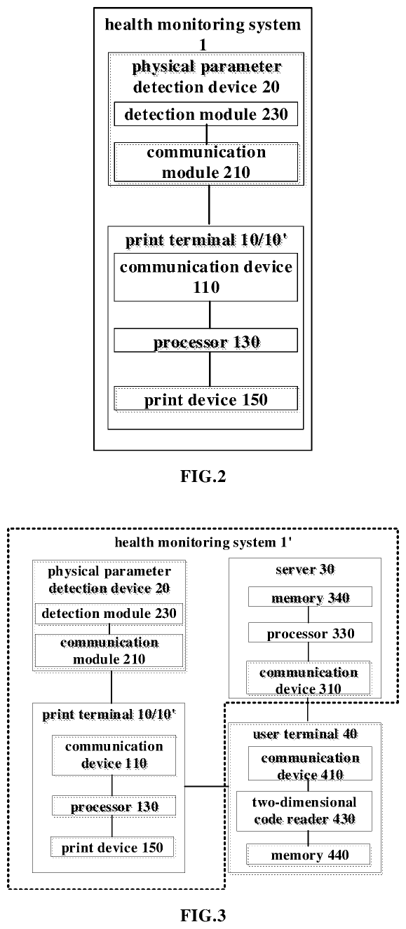

FIG. 2 is a block diagram illustrating a health monitoring system according to some embodiments of the present disclosure.

As shown in FIG. 2, the health monitoring system 1 comprises a print terminal 10/10′ and at least one physical parameter detection device 20.

Each physical parameter detection device 20 corresponds to a device two-dimensional code. The device two-dimensional code of the physical parameter detection device 20 may be displayed to a user through a display device of a print terminal. The user can select a corresponding physical parameter detection device according to actual needs. A processor 130 of the print terminal may control the operating mode of the corresponding physical parameter detection device in response to a selection on a user terminal. That is, based on the human-computer interaction between the user and the print terminal, a device response command may be sent to a selected physical parameter detection device through the communication device 110 of the print terminal. The corresponding physical parameter detection device detects a physical parameter of the human body according to a preset operating mode to obtain physical parameter information. It should be understood that the human-computer interaction between the user and the print terminal may be implemented through a display device and an input device, or may be implemented on an operation interface through a print terminal App installed on a user terminal.

Each physical parameter detection device 20 comprises a communication module 210 for communicating with the communication device 110 of the print terminal. For example, the communication module 210 may transmit the detected physical parameter information to the communication device 110 of the print terminal. As another example, the communication module 210 may receive a device response command from the communication device 110 of the print terminal.

The physical parameter detection device 20 comprises one or more devices for detecting a human blood-gas parameter, a hemodynamic parameter, and a blood chemical parameter, and one or more of a weighing device, a height detection device, a body fat detection device, a moisture detection device, a bone detection device, a blood pressure detection device, a blood lipid detection device, a heart rate detection device, and a plasma glucose detection device. Each physical parameter detection device 20 comprises at least one detection module. The detection module is used to detect a physical parameter of a human body, for example, one or more of a human blood-gas parameter, a hemodynamic parameter, a blood chemical parameter, a weight parameter, a height parameter, a body fat parameter, a moisture parameter, a bone parameter, a blood pressure parameter, a blood lipid parameter, a heart rate parameter, and a plasma glucose parameter.

In some embodiments, the health monitoring system 1 comprises a plurality of physical parameter detection devices 20 configured to obtain different physical parameter information, respectively. The processor 130 of the print terminal may generate a data two-dimensional code comprising different physical parameter information. The processor 130 of the print terminal may also generate different data two-dimensional codes for different physical parameter information.

In the above embodiment, the print terminal controls the operating mode of the physical parameter detection device, acquires physical parameter information of the user from the physical parameter detection device, and displays it to the user. The user can print a detection report in real time through the print terminal. The user can also obtain the electronic detection report by scanning the data two-dimensional code, and can save or upload the electronic detection report as needed. In this way, it is beneficial for users to obtain health detection information conveniently and flexibly. In addition, multiple physical parameter detection devices can share a single print terminal with lower cost.

FIG. 3 is a block diagram illustrating a health monitoring system according to other embodiments of the present disclosure. FIG. 3 is different from FIG. 2 in that the health monitoring system 1′ in FIG. 3 further comprises a server 30. Only the difference between FIG. 3 and FIG. 2 will be described below, and the same as in FIG. 2 will not be repeated.

As shown in FIG. 3, the server 30 comprises a communication device 310, a processor 330, and a memory 340. The server 30 is, for example, a cloud server.

The communication device 310 is configured to receive physical parameter information and basic user information provided by a user. The processor 330 is configured to process the physical parameter information and generate a health report. For example, the processor 330 uses big data to perform real-time analysis according to the feature parameter information and user basic information provided by the user terminal to generate a health report. The generated health report may be transmitted to the user terminal 40 through the communication device 310. If the analysis result shows that there is information about poor-health conditions, the generated health report may comprise warning information to indicate the poor-health conditions. The health report can also comprise corresponding health recommendations. That is, users can be provided with personalized health reports.

In some embodiments, the server 30 is further configured to create a user account by the processor 330 according to the basic user information provided by the user, and record the physical parameter information corresponding to the user account in the memory 340. The physical parameter information may be classified in the memory 340, so that visual health management can be achieved.

The user terminal 40 is, for example, a smart phone. The user terminal 40 is provided with a communication device 410, a two-dimensional code reader 430, and a memory 440. A print terminal APP may be installed on the user terminal 40, and corresponding operations such as selecting a physical parameter detection device, scanning a data two-dimensional code to acquire corresponding physical parameter information, etc., can be performed through an operation interface of the print terminal APP. The user can enter the operation interface by scanning the code and performing payment. The user terminal 40 may also be provided with an automatic identification device for automatic identification to directly retrieve account information and facilitate payment.

The user terminal 40 may read the data two-dimensional code through the two-dimensional code reader 430, and store the acquired physical parameter information in the memory 440 as historical records. The user terminal 40 may also output the physical parameter information and the basic user information obtained based on the data two-dimensional code to the server 30 through the communication device 410. The user terminal 40 may also receive a health report from the server through the communication device 410.

In the above embodiments, the physical parameter detection device and the print terminal of the health monitoring system can be installed on a large scale in public places such as shopping malls, pharmacies, hospitals, and airports. Users only need to use terminal devices such as smart phones to understand their own health data in real time, so that social costs can be saved, and the need of health monitoring and management can be met whenever and wherever possible. In addition, users can also obtain corresponding health advice services from the server to achieve early warning for health.

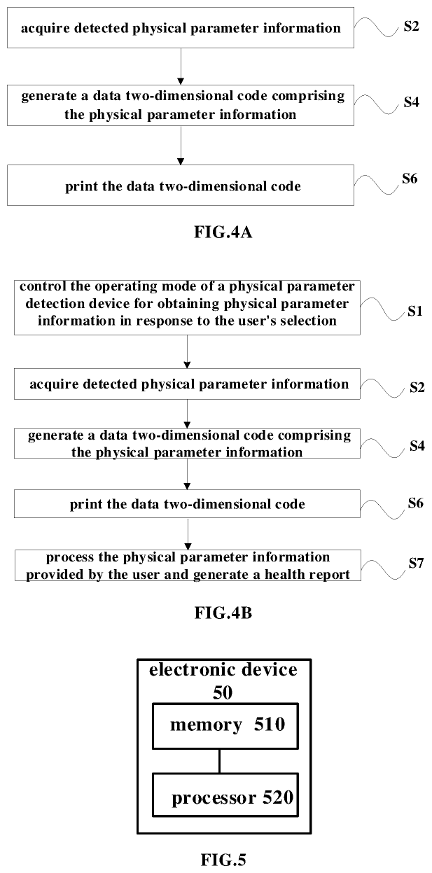

FIG. 4A is a flowchart illustrating a health monitoring method according to some embodiments of the present disclosure. As shown in FIG. 4A, the health monitoring method comprises steps S2-S6.

In step S2, detected physical parameter information is acquired. For example, the communication device of the print terminal acquires the detected physical parameter information from a physical parameter detection device. Different physical parameter information can be obtained from a variety of different physical parameter detection devices.

In some embodiments, in step S2, the print terminal further acquires basic user information through an input device. For example, the basic user information can be acquired by manual entry of a user, or the basic user information can be acquired by scanning the user's ID card or health insurance card.

In step S4, a data two-dimensional code comprising the physical parameter information is generated. For example, the processor of the print terminal converts the physical parameter information into a data two-dimensional code using a two-dimensional code generator. In some embodiments, the data two-dimensional code further comprises basic user information.

In step S6, the data two-dimensional code is printed. For example, a print device of a print terminal may print the data two-dimensional code according to the user's selection. The print device may also print a detection report comprising the physical parameter information according to the user's selection.

In some embodiments, in step S6, a display device of the print terminal may display the data two-dimensional code in response to the user's selection. The display device may also display a detection report in response to the user's selection.

In some other embodiments, in step S6, the acquired basic user information and physical parameter information may be further stored in memory of the print terminal as a historical record for retrieval.

FIG. 4B is a flowchart illustrating a health monitoring method according to other embodiments of the present disclosure. FIG. 4B is different from FIG. 4A in that the health monitoring method in FIG. 4B further comprises a step S1. Only the differences between FIG. 4B and FIG. 4A will be described below, and the same as in FIG. 4A will not be repeated.

In step S1, the operating mode of a physical parameter detection device for obtaining physical parameter information is controlled in response to the user's selection. For example, the display device of the print terminal may display a device two-dimensional code of each physical parameter detection device. The user can select a corresponding physical parameter detection device according to actual needs. The processor of the print terminal may, in response to the selection on the user terminal, send a device response command to the physical parameter detection device selected by the user through the communication device of the print terminal. The corresponding physical parameter detection device detects a physical parameter of the human body according to a preset operating mode to obtain physical parameter information.

In step S7, the physical parameter information provided by the user is processed and a health report is generated. For example, the communication device of the server receives physical parameter information and basic user information provided by the user terminal based on the data two-dimensional code. The processor of the server analyzes the user's physical parameter information and user basic information in real time using big data technology to generate a health report. The generated health report can be sent to the user terminal through the communication device of the server. If the analysis result shows that there is information about poor-health conditions, the generated health report may comprise warning information to remind the poor-health conditions. The health report can also comprise corresponding health recommendations.

In some embodiments, the processor of the server creates a user account according to the basic user information provided by the user, and records the physical parameter information corresponding to the user account in memory. The physical parameter information may be classified in the memory, so that visual health management can be achieved.

FIG. 5 is a block diagram illustrating an electronic device according to some embodiments of the present disclosure.

As shown in FIG. 5, the electronic device 50 comprises a memory 510 and a processor 520 coupled to the memory 510. The memory 510 is configured to store instructions for executing a corresponding embodiment of the health monitoring method. The processor 520 is configured to perform one or more steps of the health monitoring method in any of the embodiments of the present disclosure based on the instructions stored in the memory 510.

It should be understood that one or more steps in the foregoing health monitoring method may be implemented by a processor, and may be implemented in any of software, hardware, firmware, or a combination thereof.

In addition to the health monitoring method and device, the embodiments of the present disclosure may also take the form of a computer program product implemented on one or more non-volatile storage media containing computer program instructions. Therefore, an embodiment of the present disclosure further provides a computer-readable storage medium having computer instructions stored thereon, which, when executed by a processor, implement one or more steps of the health monitoring method in any of the foregoing embodiments.

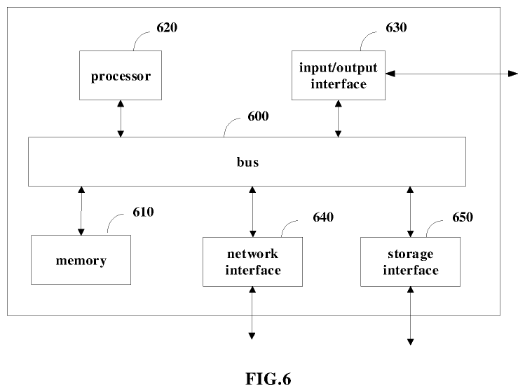

FIG. 6 is a block diagram illustrating a computer system for implementing some embodiments of the present disclosure.

As shown in FIG. 6, the computer system may be in the form of a general-purpose computing device, and the computer system may be used to implement the print terminal of the foregoing embodiment. The computer system comprises a memory 610, a processor 620, and a bus 600 connecting different system components.

The memory 610 may comprise, for example, a system memory, a non-volatile storage medium, and the like. The system memory stores, for example, an operating system, application programs, a boot loader (Boot Loader), and other programs. The system memory may comprise a volatile storage medium such as random access memory (RAM) and/or cache memory. The non-volatile storage medium stores, for example, instructions for executing a corresponding embodiment of the display method. The non-volatile storage medium comprises, but not limited to, magnetic disk storage, optical storage, flash memory, and the like.

The processor 620 may be implemented by discrete hardware components such as a general-purpose processor, a digital signal processor (DSP), an application specific integrated circuit (ASIC), a field programmable gate array (FPGA), or other programmable logic devices, discrete gates, or transistors. Accordingly, each device such as the judgment device and the determination device may be implemented by a central processing unit (CPU) running instructions that execute the corresponding steps, or may be implemented by a dedicated circuit that executes the corresponding steps.

The bus 600 may has any of a variety of bus structures. For example, these structures comprise, but are not limited to, an Industry Standard Architecture (ISA) bus, a Micro Channel Architecture (MAC) bus, and peripheral component interconnects (PCI) bus.

The computer system may further comprise an input-output interface 630, a network interface 640, a storage interface 650, and the like. These interfaces 630, 640, 650, the memory 610 and the processor 620 may be connected through a bus 600. The input/output interface 630 may provide a connection interface for input/output devices such as a display, a mouse, and a keyboard. The network interface 640 provides a connection interface for various networked devices. The storage interface 640 provides a connection interface for external storage devices such as a floppy disk, a flash disk, or an SD card.

So far, the various embodiments of this disclosure have been described in detail. In order to avoid shielding the idea of this disclosure, some of the details well known in the art are not described. Those skilled in the art can fully understand how to carry out the technical solutions disclosed herein according to the above description.

Although some specific embodiments of this disclosure have been described in detail by way of examples, those skilled in the art should understand that the above examples are for illustrative purposes only, but not for limiting the scope of this disclosure. Those skilled in the art should understand that the above embodiments can be modified or some technical features can be equivalently replaced without departing from the scope and spirit of this disclosure. The scope of this disclosure is limited by the attached claims.

What is claimed is:

1. A print terminal, comprising:
a communication device configured to acquire detected physical parameter information;
a processor configured to generate a data two-dimensional code comprising the physical parameter information; and
a printing device configured to print the data two-dimensional code.

2. The print terminal according to claim 1, further comprising an input device configured to obtain basic user information, wherein the generated data two-dimension code comprises the basic user information.

3. The print terminal according to claim 1, further comprising:
a display device configured to display the data two-dimensional code.

4. The print terminal according to claim 1, wherein the print device is configured to print a detection report comprising the physical parameter information.

5. The print terminal according to claim 2, further comprising:
a memory configured to store the acquired basic user information and the physical parameter information.

6. The print terminal according to any one of claims 1 to 5, wherein the print terminal is a handheld smart printer.

7. A health monitoring system, comprising the print terminal according to any one of claims 1 to 6.

8. The health monitoring system according to claim 7, further comprising:
at least one physical parameter detection device configured to detect a human body to obtain the physical parameter information.

9. The health monitoring system according to claim 8, wherein:
the at least one physical parameter detection device comprises a plurality of physical parameter detection devices configured to obtain different physical parameter information respectively; and
the processor of the print terminal is configured to generate different data two-dimensional codes for different physical parameter information.

10. The health monitoring system according to claim 8, wherein:
the at least one physical parameter detection device comprises a plurality of physical parameter detection devices configured to obtain different physical parameter information respectively; and
the processor of the print terminal is configured to generate a data two-dimensional code comprising different physical parameter information.

11. The health monitoring system according to claim 8, wherein the display device of the print terminal is configured to display a device two-dimensional code of the at least one physical parameter detection device.

12. The health monitoring system according to any one of claims 8 to 11, wherein the communication device of the print terminal communicates with the at least one physical parameter detection device via Bluetooth.

13. The health monitoring system according to any one of claims 8 to 11, wherein the processor of the print terminal is configured to control an operating mode of the at least one physical parameter detection device in response to a user's selection.

14. The health monitoring system according to claim 7, further comprising:
a server configured to process the physical parameter information provided by the user terminal based on the data two-dimensional code, and generate a health report.

15. The health monitoring system according to claim 14, wherein the server is further configured to: create a user account according to the basic user information provided by the user terminal based on the data two-dimensional code, and record physical parameter information corresponding to the user account.

16. A health monitoring method, comprising:
acquiring detected physical parameter information;
generating a data two-dimensional code comprising the physical parameter information; and
printing the data two-dimensional code.

17. The health monitoring method according to claim 16, further comprising:
displaying the data two-dimensional code in response to a user's request.

18. The health monitoring method according to claim 16, further comprising: in response to a user's selection, controlling an operating mode of the physical parameter detection device for obtaining the physical parameter information.

19. The health monitoring method according to claim 16, further comprising: processing the physical parameter information provided by the user terminal based on the data two-dimensional code, and generating a health report.

20. A computer-readable storage medium on which a computer program is stored, which when executed by a processor implements one or more steps of the health monitoring method according to any one of claims 16 to 19.
